(19) United States
(12) Patent Application Publication
(10) Pub. No.: US 2019/0008195 A1
(43) Pub. Date: Jan. 10, 2019

(54) COMPOSITION FOR IMPROVING MOUTHFEEL

(71) Applicant: NESTEC S.A., Vevey (CH)

(72) Inventors: Sherri Ann Slocum, Plymouth, MN (US); Norman Alan Greenberg, New Hope, MN (US); Sarah Holton, Minneapolis, MN (US)

(21) Appl. No.: 16/066,932

(22) PCT Filed: Dec. 22, 2016
(86) PCT No.: PCT/EP2016/082316

§ 371 Date: Jun. 28, 2018

Related U.S. Application Data
(60) Provisional application No. 62/273,605, filed on Dec. 31, 2015.

(51) Int. Cl. A23L 29/238; A23L 29/25; A23L 33/00; A61K 31/736; A61K 47/26; A61K 47/42; A61K 36/48

(52) U.S. Cl. CPC A23L 29/238; A23L 29/25; A23L 33/40; A23L 33/30; A23V 2002/00; A61K 47/26; A61K 47/42; A61K 36/48; A61K 31/736

(57) ABSTRACT

Disclosed are compositions useful for improving or enhancing the organoleptic properties, especially mouthfeel, of nutritional compositions. The composition contains a combination of partially hydrogenated guar gum (PHGG) and acacia gum (AG). These may also be used to promote a healthy gut flora and treat or prevent digestive diseases when included in a nutritional composition.

### FIELD OF THE INVENTION

The present invention relates to a composition which is useful for improving or enhancing the organoleptic properties, in particular mouthfeel, of nutritional compositions. The invention also relates to a nutritional formulation containing the composition.

### BACKGROUND

Irritable bowel syndrome (IBS) is a common chronic gastrointestinal (GI) disorder, which is estimated to affect around 5-20% of the general population in developed countries. IBS is a disorder affecting the large intestine, and typically accounts for more than half of all patients with digestive complaints. IBS is a chronic condition that presents with disparate clinical symptoms such as, e.g. abdominal pain, diarrhoea, constipation, alternating bouts of diarrhoea and constipation, other disturbed bowel habits, bloating, gas, abdominal discomfort and distension, excessive mucus in the stool, rectal bleeding and weight loss. In many cases, the symptoms of IBS can be extremely troublesome, having a severe negative impact on the quality of life of the patient.

The causes and pathophysiology of IBS are not well understood, and a number of factors are considered to play a role (R. Spiller, et al.: Gut 2007, 56, 1770-1798). In IBS patients, the intestinal muscles may contract in an abnormal manner. For example stronger contractions may cause the symptoms of gas, bloating and diarrhoea, and weaker contractions may cause abdominal distension and constipation. Often, IBS is associated with a heightened sensitivity to visceral pain perception, known as peripheral sensitization. This sensitization involves a reduction in the threshold and an increase in the gain of the transduction processes of primary afferent neurons, attributable to a variety of mediators such as monoamines (e.g., catecholamines and indoleamines), substance P, and a variety of cytokines and prostanoids such as E-type prostaglandins. Also implicated in the etiopathology of IBS is intestinal motor dysfunction, which leads to abnormal handling of intraluminal contents and/or gas. Psychological factors are also thought to contribute to IBS symptoms appearing in conjunction with, if not triggered by, disturbances including depression and anxiety. Infection and inflammation due to pathogenic viruses and bacteria in the GI tract may be a causative or exacerbating factor contributing to IBS in some patients. Nutritional factors (e.g. insufficient fibre intake, excessive caffeine intake—caffeine is known to be a GI stimulant), high fat consumption, food intolerance (e.g. milk, wheat and eggs are often implicated in IBS), and carbohydrate malabsorption (especially fructose, lactose and sorbitol) may play a large part in influencing motility, and may give rise to IBS symptoms.

Treatment for IBS depends on the severity and symptoms. The treatment and management may involve lifestyle changes including dietary changes such as restricting or avoiding certain foods, increasing dietary fibre intake, avoiding particular food additives, and exercise, and/or drug therapy. Typically, a multi-modal approach is utilised in managing and controlling IBS symptoms.

The role of diet in the management of IBS has been extensively investigated. In some IBS patients, symptoms have been attributed to an intolerance of particular sugars or a malabsorption of carbohydrates. Thus, in some cases, management of IBS symptoms has been achieved by exclusion diets.

One such exclusion diet is the so-called “low FODMAP” diet. In such a diet, the intake of short chain carbohydrates that are slowly absorbed or indigestible and not absorbed during passage through the small intestine are restricted, and are rapidly fermented by gut bacteria, resulting in the release of gases and the associated bloating symptoms commonly experienced in IBS patients. These carbohydrates are collectively referred to as FODMAPs—fermentable oligosaccharides (mainly fructans, galacto-oligosaccharides), disaccharides, monosaccharides and polyols (see Gibson, P. R., and Shepherd, S. J. —Aliment. Pharmacol. Ther. 2005; 21: 1399-1409). A low FODMAP diet generally avoids foods high in these carbohydrates and replaces these with foods low in FODMAPs in order to control symptoms. A growing number of studies have reported efficacy in the use of a low FODMAP diet for the management of IBS (Ong, D. K., et al.: J Gastroenterol Hepatol 2010; 25:1366-1373; Halmos E. P. et al., Gastroenterology 2014; 146:67-75, e5, Staudacher H. M., et al.: J Hum. Nutr. Diet 2011; 24: 487-495).

FODMAPs are naturally found in a large number of foods. FODMAPs are generally either indigestible in the gut due to the absence of appropriate enzymes, or slowly absorbed in the small intestine. For example, indigestible oligosaccharides include: fructooligosaccharides (fructans) which are found in onion, garlic, artichoke, leek, shallot, cereals such as wheat, barley and rye, and inulin, and galacto-oligosaccharides (GOS), which are found in nuts, legumes, kidney beans, lentils, chickpeas, soy beans, and soy products; disaccharides such as lactose, found in milk and milk products such as cheese and yoghurt; monosaccharides such as fructose, which is found in a large range of fruits including apple, pear, watermelon and mango, honey and high fructose corn syrup; sugar alcohols (polyols), which include sorbitol, which is present in avocado and apricots, and mannitol, which is found in mushrooms and cauliflower; and polyols (sugar alcohols) including sorbitol, which are found in apricots, cherries, nectarines, pears, avocados, plums, lychee, and mannitol found in mushrooms, snow peas, and cauliflower.

A typical low FODMAP food contains:

<0.15 g fructose in excess of glucose/100 g,

<3 g fructose per serving regardless of glucose, and

<0.2 g of fructans per serving (except for grains, nuts and seeds, which is <0.3 g per serving).

In addition, an increased intake of dietary fibre (particularly soluble fibre) is recommended.

FODMAPS are water-soluble, and due to their osmotic properties, attract water into the small bowel and colon. In the large bowel, they are fermented by bacteria, releasing gases that lead to bloating and associated IBS symptoms in susceptible patients. A low FODMAP diet has been shown to be effective for improving the gut symptoms, with around 70% of IBS patients experiencing rapid improvement in gut symptoms.

However, the long-term exclusion or limitation of high FODMAP foods such as nutrient-rich vegetables, fruits and dairy products, can result in the need to provide nutritional supplementation. Calcium is a particularly important nutrient which may be lacking in a low FODMAP diet due to the exclusion of dairy-based foods.

It is well-recognised that consumption of dietary fibre is an essential part of maintaining a healthy digestive tract. For example, the World Health Organisation (WHO) recommends a daily consumption of 25 g or more of dietary fibre. Many IBS patients are deficient in dietary fibre intake, and thus are typically recommended to increase intake of fibre. However, certain high fibre foods should be restricted or eliminated from low FODMAP diets since some high fibre foods are also high in FODMAPS. For example, the types of fibre found in plant-based foods such as beans, fruits, vegetables and wholegrains (typically insoluble fibre components) contain complex carbohydrates that cannot be digested. Hence, a low FODMAP diet may not provide sufficient dietary fibre (especially soluble fibre) intake.

A particular problem with nutritional supplement formulations, especially for low FODMAP diets, arises due to the restriction or exclusion of the problematical short chain carbohydrates. These short chain carbohydrates are typically responsible for the organoleptic properties and sensory properties of the nutritional composition. A reduction of such carbohydrates and total solid content has a detrimental effect on these properties. For example, the compositions may suffer from a mouthfeel which is thin and lacks body. A poor mouthfeel experience, particularly in nutritional compositions, is associated with decreased consumer acceptability and patient compliance.

Therefore, there is a need to provide nutritional compositions having improved or enhanced organoleptic and sensory properties, particularly mouthfeel. However, many of the known food additives used to improve texture and mouthfeel are unstable to UHT processing, and may be incompatible with particular dietary requirements, such as low FODMAP diets. Although fibres can be used in nutritional compositions as texturants, the amount needed to provide a nutritionally significant amount of fibre would typically increase the viscosity to an unacceptable level. There is a further need to provide nutritional compositions including sufficient levels of fibre and which are suitable for patients on a low FODMAP diet, and which provide a satisfactory sensory experience, thereby improving patient compliance.

### SUMMARY OF THE INVENTION

The present invention is based on the surprising finding that a combination of a water-soluble dietary fibre and an arabinogalactan, i.e. partially hydrolysed guar gum (PHGG) and acacia gum (AG), is highly effective at enhancing the mouthfeel of nutritional compositions. In particular, the combination of PHGG and AG are able to enhance the mouthfeel more effectively than the same amount of the individual components. The combination of the invention is suitable for a low FODMAP diet, and hence is particularly suitable for use in nutritional formulations such as for IBS patients.

Advantageously the compositions are stable, and thus are particularly suitable for preparing nutritional compositions which typically require heat sterilization, for example, any heat treatment such as to remove, reduce the content of, or otherwise reduce the activity of pathogenic microorganisms—including, e.g. UHT sterilization or pasteurization and autoclaving.

A further advantage is that the combination of the invention provides a source of soluble dietary fibres, which are a highly beneficial dietary component, and which are of particular importance in the management of functional GI disorders such as IBS.

The present invention provides a combination of partially hydrolysed guar gum (PHGG) and acacia gum (AG) for improving mouthfeel of a nutritional composition. In particular, the present invention provides a combination of acacia gum (AG) and partially hydrolysed guar gum (PHGG) for improving mouthfeel of a nutritional composition, wherein AG and PHGG are present in a weight ratio of AG:PHGG of about 5:1 to about 1:5.

The combination may further comprise a starch hydrolysate, preferably wherein the starch hydrolysate is maltodextrin. Thus, in some embodiments, the present invention provides the use of a combination of partially hydrolysed guar gum, acacia gum and a starch hydrolysate, preferably maltodextrin, for improving mouthfeel of a nutritional composition.

In preferred embodiments, the nutritional composition is in the form of a liquid or in the form of a powder for reconstitution into a liquid.

In another aspect, the present invention provides a nutritional composition comprising a combination of acacia gum (AG) and partially hydrolysed guar gum (PHGG). In another aspect, the present invention provides a nutritional composition comprising a combination of PHGG, AG and a starch hydrolysate, preferably maltodextrin. The nutritional compositions are preferably in the form of a liquid or a powder for reconstitution into a liquid.

The present invention further provides a method of promoting GI microbiota balance and health comprising administering an effective amount of the nutritional composition to an individual.

In a further aspect, the present invention provides a nutritional supplement as described in any aspect or embodiment herein, for the treatment or prophylaxis of a functional GI disorder or a digestive disorder, preferably irritable bowel syndrome or functional dyspepsia, and more preferably irritable bowel syndrome.

In a yet further aspect, the present invention provides a nutritional supplement as described in any aspect or embodiment herein, for the treatment or prophylaxis of symptoms of a GI tract disorder or a digestive disorder, preferably selected from: impaired GI motility, dyspepsia, abdominal pain, abdominal cramp, diarrhoea, constipation, disturbed bowel habits, bloating, excess gas, abdominal discomfort and abdominal distension.

### DETAILED DESCRIPTION OF THE INVENTION

Unless otherwise indicated, references to % relate to weight %.

As used herein the term “soluble fibre” refers to a fibre that attracts water and turns to gel during digestion, thereby slowing digestion. Soluble fiber is found in, for example, oat bran, barley, nuts, seeds, beans, lentils, peas, and some fruits and vegetables. It is also found in psyllium, a common fiber supplement. Insoluble fiber is found in foods such as wheat bran, vegetables, and whole grains. It adds bulk to the stool and appears to help food pass more quickly through the stomach and intestines.

As used herein, the term “low solids” in relation to the nutritional compositions, particularly refer to liquid (aqueous compositions) having a total solids content of 10-19 wt %, and preferably 15-18 wt %. The term “solids” in the context of the present invention refers to the non-water components of the composition. Thus, the term “total solids” refers to the whole content of the composition minus water. In particular, the total solids relates to the dry matter left after removing the free and loosely bound moisture. The total solids content in a composition may be determined, for exmple, by drying the composition at 70-100° C. under reduced pressure, such as in a vacuum oven (e.g. according to Association of Analytical Communities (AOAC International), Official Methods of Analysis 925.09 or 926.08.

As used herein, the term “high solids” relation to nutritional compositions refers to a total solids content of 20-28 wt %, and preferably 21.5-24 wt %.

As used herein, the term “low sugar” in relation to nutritional compositions refers to composition having a sugar content of about 7 wt % or less, about 5 wt % or less, particularly about 3 wt % or less, about 2 wt % or less or about 1 wt % or less. Preferably, “low sugar” refers to nutritional compositions having a sugar content of about 1 to about 7 wt %, about 1 to about 6 wt %, about 1 to about 5 wt %, about 2 to about 5 wt % or about 2 to about 4 wt %.

As used herein, the term “low energy density” in relation to nutritional compositions refers to compositions having an energy density of less than 1 kcal/g. For example, low energy density nutritional compositions may have an energy density of about 0.3 to about 0.95 kcal/g, about 0.4 to about 0.9 kcal/g, about 0.45 to about 0.88 kcal/g, about 0.5 to about 0.85 kcal/g, about 0.55 to about 0.8 kcal/g, about 0.6 to about 0.75 kcal/g, or about 0.6 to about 0.7 kcal/g.

“FODMAPs” are described in Gibson, P. R., and Shepherd, S. J. —Aliment. Pharmacol. Ther. 2005; 21: 1399-1409, in particular Table 2, page 1402). Essentially FODMAPS comprise 5 main dietary components—fructose (typically from fruits, honey, high fructose corn syrup), fructans (fructooligosaccharides/oligofructose—typically from wheat and onions), lactose (typically from milk/milk products, polyols (such as sorbitol, xylitol, mannitol, maltitol—typically from apples, pears, plums, reduced calorie sweeteners) and galactooligosaccharides (GOS) (such as raffinose, stachyose, typically from legumes, beans, cabbage, Brussels sprouts, onions).

As used herein, the term “mouthfeel” in relation to a nutritional composition refers to the sensory and tactile properties of the nutritional composition perceived when the composition contacts the mouth cavity and surfaces. The sensory and tactile properties include the texture, thickness, consistency and body. An improvement or enhancement in the mouthfeel may refer to an improvement or enhancement in at least one sensory/tactile property, such as thickness and/or body. It will be appreciated that such in mouth sensory and tactile properties may be independent of properties such as viscosity measured by an instrument. For example, an improved or enhanced mouthfeel can be achieved without a detrimental impact (particularly an unacceptable increase) on viscosity. The improved mouthfeel may be examined by a taste panel trained on sensory attributes.

Partially Hydrolysed Guar Gum (PHGG)

PHGG is a water-soluble dietary fibre which is obtained from guar gum. Guar gum is a high molecular weight polysaccharide composed of galactomannans, and is obtained from grinding the endosperm of guar beans (Cyamopsis tetragonolobus L). PHGG is produced by partial hydrolysis of guar gum, typically via controlled enzyme hydrolysis, typically using β-endo-mannanase. Following hydrolysis the product may be sterilized and spray dried to form a powder.

Structurally, guar gum comprises long, straight chains of α-D-mannopyranosyl units linked via β-D-(1-4)-glycosidic linkages. PHGG has the same chemical structure and mannose:galactose ratio as guar gum (approximately 2:1), but the hydrolysis reduces the chain length of the guar gum to less than 10% of the original (intact) guar gum. The average molecular weight is typically reduced by around 10% of intact guar gum. The molecular weight of PHGG typically ranges between 1-100 kDa. PHGG is less viscous than guar gum.

Preferably, a 1 wt % aqueous solution of guar gum may have a viscosity of about 2,000 to about 6,000 mPa·s, typically about 3000 to about 6000 mPa·s, or about 3000 to about 5000 mPa·s, as measured at 25° C. by a rheometer. Preferably a 5 wt % aqueous solution of the PHGG employed in the present invention may have a viscosity of about 5 to about 15 mPa·s, more preferably about 6 to about 14, and most preferably about 7 mPa·s to about 12 mPa·s, as measured at 5° C. by a rheometer.

PHGG has been reported as having a positive effect on diarrhoea and constipation (Homann, H, H., et al.: Journal of Parenteral and Enteral Nutrition 1994, 18, 486-490 and Takahashi, H, et al.: Journal of Nutritional Science and Vitaminology 1994, 40, 251-259. PHGG has been extensively used as a food additive, and as a nutritional additive without altering the rheology, taste, texture and colour of final products (Yoon, S.-J. et al.: Journal of Clinical Biochemistry and Nutrition 2008, 42, 1-7). According to Heini, A. F. et al.: (International Journal of Obesity 1998, 22, 906-909), PHGG could be added as a soluble fibre to a nutritional drink without the subject's conscious awareness, and hence had no effect on mouthfeel or texture.

Acacia Gum (AG)

Acacia gum (AG), also known as gum Acacia, Gum Arabic (GA) or Indian gum, is a natural, non-viscous, highly water-soluble, fibre belonging to the complex arabinogalactan family. AG is obtained as an exudate from the stems and branches of certain Acacia trees (Leguminosae—primarily A. Senegal and A. seyal). AG is a complex highly branched, high molecular weight polysaccharide comprised mainly of arabinose, galactose, rhamnose, and glucuronic acid units in an approximate molar ratio of about 3:3:1:1. AG has an average molecular weight of between 200 and 400 kDa. AG is composed of three different fractions, i.e., about 1% glycoprotein, about 1-10% arabinogalactan-protein, and about 90-99% arabinogalactan. Viscosities of AG vary depending on the source, and typically range from 12-18 ml/g.

AG is a soluble dietary fibre, which is slowly fermented compared to other soluble fibres. AG has been reported to improve symptoms of diarrhoea in animal studies.

Preferably the AG employed in the present invention has an average molecular weight of about 200,000 to about 400,000, more preferably about 250,000 to about 350,000, and most preferably about 275,000 to about 320,000.

Maltodextrins

Maltodextrin is a water-soluble polysaccharide formed by partial hydrolysis of starch, typically by the action of acid and/or an enzyme. Maltodextrin can be prepared from any starch source, such as wheat or corn. The resulting hydrolysis product is typically purified and spray-dried to form a powder. Chemically, maltodextrin comprises α-D-glucose units linked with glycosidic (1→4) bonds. Maltodextrins consist of mixture of saccharides, maltose and a mixture of oligosaccharides and polysaccharides (e.g. maltotriose and maltotetraose).

Maltodextrins are usually classified by dextrose equivalent (DE). DE is an inverse measure of the number of anhydro α-D-glucose units. Thus a maltodextrin of having a lower dextrose equivalent (e.g. DE 5) has a lower extent of starch hydrolysis and hence higher average molecular mass, whereas a maltodextrin having a higher DE (e.g. DE 20) has a greater extent of starch hydrolysis and hence a lower number glucose molecules, and hence a lower average molecular mass).

Probiotics

The nutritional composition according to any aspect or embodiment of the present invention may additionally include probiotics. As used herein, probiotics are food-grade microorganisms (alive, including semi-viable or weakened, and/or non-replicating), metabolites, microbial cell preparations or components of microbial cells that could confer health benefits on the host when administered in adequate amounts, more specifically, that beneficially affect a host by improving its intestinal microbial balance, leading to effects on the health or well-being of the host. See, Salminen S, Ouwehand A. Benno Y. et al, “Probiotics: how should they be defined?” Trends Food Sci. Technol. 1999: 10, 107-10. In general, it is believed that these microorganisms inhibit or influence the growth and/or metabolism of pathogenic bacteria in the intestinal tract. The probiotics may also activate the immune function of the host.

Non-limiting examples of probiotics include Aerococcus, Aspergillus, Bacillus, Bacteroides, Bifidobacterium, Candida, Clostridium, Debaromyces, Enterococcus, Fusobacterium, Lactobacillus, Lactococcus, Leuconostoc, Melissococcus, Micrococcus, Mucor, Oenococcus, Pediococcus, Penicillium, Peptostrepococcus, Pichia, Propionibacterium, Pseudocatenulatum, Rhizopus, Saccharomyces, Staphylococcus, Streptococcus, Torulopsis, Weissella, or combinations thereof. Preferred examples of suitable probiotics which may be beneficial, particularly for alleviating the symptoms of IBS include: Lactobacillus, Streptococcus and Bifidobacterium species, such as Lactobacillus acidophilus, Lactobacillus plantarum, Bifidobacterium breve, Bifidobacterium infantis, Bifidobacterium animalis, Lactobacillus rhamnosus, Lactobacillus casei, Bifidobacterium longum, Lactobacillus bulgaricus, and Streptococcus salivarius ssp. Thermophiles.

Combinations for Improving Mouthfeel

The inventive combination for improving mouthfeel of a nutritional composition comprises acacia gum (AG) and partially hydrolysed guar gum (PHGG). Thus, the present invention provides, in one aspect, a combination of acacia gum (AG) and partially hydrolysed guar gum (PHGG) for improving mouthfeel of a nutritional composition. The combination of AG and PHGG can be used to improve the mouthfeel particularly of a liquid nutritional supplement. Preferably the liquid nutritional supplement has low solids content.

The AG and PHGG are present in the nutritional composition in a weight ratio of AG:PHGG of about 5:1 to about 1:5, about 5:1 to about 1:3, about 4:1 to about 1:2, about 3.5:1 to about 1:2, about 3.25:1 to about 1:1, about 3.1 to about 1.25:1, about 2.5:1 to about 1.5:1 or about 2.25:1 to about 1.25:1. Preferably, the weight ratio of AG to PHGG is greater than 1:1. The combination of AG and PHGG to be added to the nutritional composition is preferably about 0.6 to about 2.5 wt %, about 0.7 to about 2 wt %, about 0.8 to about 1.7 wt %, or about 1 to about 1.5 wt % of the nutritional composition.

Preferably the PHGG employed in the present invention has a molecular weight of about 15 to about 35,000 kDa, more preferably about 20 to about 20 kDa. Preferably a 5 wt % aqueous solution of the PHGG may have a viscosity of about 5 to about 15 mPa·s, more preferably about 6 to about 14, and most preferably about 7 mPa·s to about 12 mPa·s, as measured at 5° C.

Preferably, the AG employed in the present invention has a molecular weight of about 200,000 to about 400,000, more preferably about 250,000 to about 350,000, and most preferably about 275,000 to about 320,000. Preferably, a 25 wt % aqueous solution of the AG has a viscosity of between about 45 to about 150 mPa·s, more preferably about 50 mPa·s to about 120 mPa·s, and most preferably about 60 mPa·s to about 100 mPa·s at 20° C.

In some embodiments of the invention, a starch hydrolysate may be further included in the combination in order to provide further enhancement or improvement of mouthfeel. Preferably the starch hydrolysate is maltodextrin or rice dextrin, preferably maltodextrin. The source of starch used to prepare the starch hydrolysate can include those selected from the group consisting of: rice, corn, tapioca, barley, pea and sorghum.

Thus, the present invention further provides a combination of AG, PHGG and a starch hydrolysate, preferably maltodextrin, for improving mouthfeel of a nutritional composition. Suitable starch hydrolysates include hydrolysates prepared from a starch source such as rice, corn, tapioca, barley, pea and sorghum. Maltodextrin is a preferred starch hydrolysate. Preferably, the maltodextrin has a dextrose equivalent (DE) integer value of 5-30, preferably 5-25, and more preferably 5-20. Maltodextrins of DE 16 or 18 are particularly preferred.

When a starch hydrolysate is used in combination with AG and PHGG, the ratio of starch hydrolysate, preferably maltodextrin, to total AG and PHGG is preferably: about 5:1 to about 1:5, about 4:1 to about 1:3 about 4:1 to about 1:2, about 3.5:1 to about 1:1, about 3:5:1 to about 1.5:1, or about 3:1 to about 2:1.

Whilst the combinations of AG and PHGG, or AG, PHGG and starch hydrolysate are particularly suitable for improving or enhancing mouthfeel of nutritional compositions, the combinations are especially useful for improving the mouthfeel of nutritional compositions which have a low sugars or low solids content as defined herein. Such compositions often suffer from poor mouthfeel, such as poor thickness and body, which are attributable to the low solids content. Merely increasing the viscosity of such compositions does not necessary correlate with improving the mouthfeel (for example by improving the perception of thickness and/or body as achieved by the present invention). The combinations of the present invention are particularly suitable for enhancing the mouthfeel of such compositions such that they are comparable to energy-dense compositions, for example ≥1 kcal/g, such as those having an energy density of about 1 kcal/g or about 1-1.2 kcal/g.

For example, the present invention is particularly useful for enhancing or improving the mouthfeel of nutritional compositions having a “low energy density” (typically less than 1 kcal/g). In particular, the combinations of the present invention are especially useful for nutritional compositions having an energy density of about 0.3 to about 0.95 kcal/g, about 0.4 to about 0.9 kcal/g, about 0.45 to about 0.88 kcal/g, about 0.5 to about 0.85 kcal/g, about 0.55 to about 0.8 kcal/g, about 0.6 to about 0.75 kcal/g, or about 0.6 to about 0.7 kcal/g. The combinations according to the present invention are also useful for improving or enhancing the mouthfeel of nutritional compositions having a low total solids content (such as a total solids content of 10-19 wt %, and preferably 15-18 wt %). The combination is particularly suitable for enhancing the mouthfeel of such compositions to be comparable with compositions containing a high solids content, for example >21 wt % total solids, such as 20-28 wt % or particularly, 21.5-24 wt % total solids.

The solids content of the nutritional composition may comprise a combination of dietary macronutrients, in particular, protein, fat and carbohydrate, to form a nutritional supplement. Preferably, the nutritional compositions of the present invention comprises a combination of protein, fat and carbohydrate.

Preferably, the nutritional compositions may have an amount of solids from the protein, fat and carbohydrate in the range of about 9-19 wt %, preferably about 12-18 wt %, and more preferably about 14-17 wt %.

The nutritional compositions may preferably comprise an amount of carbohydrates in the range of about 10-17 wt %, about 11-16.5 wt %, about 12-16 wt % or about 13-15.5 wt %. Preferably, the amount of carbohydrates in the form of sugars in the nutritional composition is relatively low. More preferably, the amount of sugars in the nutritional composition is: about 0.8 to about 8.5 wt %, about 0.8 to about 7.5 wt %, about 0.8 to about 6.5 wt %, about 0.8 to about 5.5 wt %, or about 0.8 to about 4.5 wt %.

Preferably, when the composition contains protein, the amount of protein in the nutritional composition is about 5 to about 25 wt %, about 5 to about 15 wt % or about 5 to about 10 wt %.

Preferably, when the composition contains a fat, the amount of fat is relatively low. More preferably, the amount of fat in the nutritional composition is less than 5 wt %. is about 1 to about 5 wt %, about 1 to about 3 wt %, about 1 to about 2.5 wt %, or about 1.2 to about 2 wt %.

The nutritional compositions are preferably those suitable for low FODMAP diets, for example, as recommended for treating or reducing symptoms of IBS as described above. Thus, the combinations of AG and PHGG and optionally a starch hydrolysate as defined according to any aspect or embodiment of the present invention as described herein, are especially suitable for improving or enhancing nutritional compositions suitable for low FODMAP diets, and thus may be formulated to contain 0.5 g or less, 0.4 g or less, 0.3 g or less FODMAPs per serving.

The nutritional compositions can be in any form. In particularly preferred embodiments, the combination of AG and PHGG, and optionally starch hydrolysate (preferably maltodextrin) can be used to enhance the mouthfeel of a nutritional composition which is a liquid (i.e. a liquid nutritional supplement).

The present invention further provides the use of a combination of AG and PHGG, and optionally starch hydrolysate (preferably maltodextrin), as described in any aspect or embodiment described herein, for improving the mouthfeel of a nutritional composition, especially a liquid nutritional composition.

Nutritional Composition

The present invention further provides a nutritional composition comprising a combination of acacia gum (AG) and partially hydrolysed guar gum (PHGG) wherein AG and PHGG are present in a weight ratio of AG:PHGG of about 5:1 to about 1:5.

Preferably, in the nutritional composition, AG and PHGG are present in a weight ratio of AG:PHGG of about 5:1 to about 1:3, about 4:1 to about 1:2, about 3.5:1 to about 1:2, about 3.25:1 to about 1:1, about 3.1 to about 1.25:1, about 2.5:1 to about 1.5:1 or about 2.25:1 to about 1.25:1.

The nutritional composition may preferably contain an amount of AG and PHGG of: about 0.6 to about 2.5 wt %, about 0.7 to about 2 wt %, about 0.8 to about 1.7 wt %, or about 1 to about 1.5 wt %.

As discussed above, the nutritional composition may further comprising a starch hydrolysate, preferably wherein the starch hydrolysate is maltodextrin. The ratio of starch hydrolysate, preferably maltodextrin, to total AG and PHGG is preferably: about 5:1 to about 1:5, about 4:1 to about 1:3 about 4:1 to about 1:2, about 3.5:1 to about 1:1, about 3:5:1 to about 1.5:1, or about 3:1 to about 2:1. The starch hydrolysate, preferably maltodextrin, can be employed in the nutritional composition in an amount of: about 1 to about 8 wt %, about 2 to about 6 wt %, or about 3 to about 5 wt %.

Particularly preferred are nutritional compositions according to the present invention having a low energy density, i.e. composition having an energy density of: less than about 1 kcal/g, about 0.4 to about 0.98 kcal/g, about 0.55 to about 0.95 kcal/g, about 0.5 to about 0.9 kcal/g, about 0.6 to about 0.85 kcal/g, about 0.65 to about 0.8 kcal/g, or about 0.6 to about 0.75 kcal/g.

The present invention further provides a nutritional composition having a low total solids content, i.e.: about 12 to about 20 wt %, about 14 to about 19 wt %, about 16 to about 18.5 wt % or about 15 to about 18 wt %.

Surprisingly, the addition of a combination of AG and PHGG and optionally a starch hydrolysate to a nutritional composition having low energy density and/or low solids content, is able to improve or enhance the mouthfeel such that the compositions are comparable to the high energy density/high solids content counterpart compositions. Moreover, the improvement in mouthfeel using the combination of AG and PHGG has been found to be greater when using the combination of the invention, as compared to using the same amount of AG or PHGG alone.

The solids in the nutritional composition may comprise macronutrients, preferably selected from protein, fat and carbohydrate. The composition may be a nutritionally complete formula, for example including a source of protein, carbohydrate and fat.

Any suitable dietary protein may be used. Preferably dietary proteins suitable for a low FODMAP diet are used. For example, vegetable proteins (such as soy protein, rice protein, and pea protein); mixtures of free amino acids; or combinations thereof. It is preferable to avoid proteins from meat sources. Particulary preferred are milk proteins, for example in the form of milk protein concentrate (MPC) and/or caseinate (e.g. sodium caseinate). Milk proteins having a reduced lactose content are particularly preferred for low FODMAP diets. Soy proteins may also be used. For low FODMAP diets, purified soy protein containing reduced or no galactans are preferred. Combinations of soy protein with milk proteins may also be used.

The composition may also contain a source of carbohydrates and a source of fat.

Suitable fats for a low FODMAP diet are monounsaturated fats (e.g. nut, vegetable, olive and sunflower oils), and polyunsaturated fats (e.g. soybean, corn, safflower, flaxseed and fish oils). The source of fat may comprise at least one omega-3 polyunsaturated fatty acid, for example those found in fish oils, especially eicosapentaenoic acid (EPA), and docosahexaenoic acid (DHA). The fat preferably provides about 40 wt % or less of the energy, preferably about 35 wt % or less of the energy, more preferably about 25 wt % or less of the energy, or particularly about 20 wt % or less of the energy of the nutritional supplement to meet dietary macronutrient guidleines.

It is preferred that the nutritional composition is low in fat, i.e. preferably less than about 4 wt % fat, less than about 3 wt %, more preferably less than about 2 wt % fat, most preferably less than about 1.8 wt % fat. More preferably, the nutritional compositions contain fat in the range of about 0.8 to about 3 wt %, about 0.8 to about 2.8 wt %, about 0.8 to about 2.4 wt %, or about 1 to about 1.8 wt %. Preferably, the fat provides about 15 to about 45%, about 18 to about 36%, about 20 to about 30%, or about 20 to about 25% of the total energy of the nutritional composition.

A source of carbohydrate may be added to the nutritional composition in addition to the PHGG, AG and, where present, maltodextrins. The carbohydrate (i.e. not including fibre) preferably provides about 4 to about 70%, about 4 to about 61%, about 4 to about 55%, about 10 to about 50%, about 20 to about 40% or about 20 to about 35% of the total energy of the nutritional composition. Any suitable low FODMAP carbohydrate may be used, for example maltodextrin or other polymers of glucose. The amount of carbohydrates in the nutritional composition is preferably in the range of about 1-16 wt %, about 1-15, about 3-14 wt %, about 4-12 wt % or about 5-10 wt %. The amount of carbohydrates in the form of sugars in the nutritional composition is preferably: about 0.7 to about 7.0 wt %, about 0.7 to about 7.0 wt %, about 0.7 to about 6.0 wt %, about 0.7 to about 5.0 wt %, or about 0.7 to about 4.0 wt %.

The amount of protein in the nutritional composition is: preferably in the range of: about 4 to about 24 wt %, about 4 to about 14 wt %, or about 4 to about 9 wt %. Preferably, the protein provides about 15 to about 65%, about 20 to about 53%, about 25 to about 45%, about 25 to about 40% or about 30 to about 40% of the total energy of the nutritional composition.

Preferably, the amount of solids from the protein, fat and carbohydrate in the nutritional composition is about 9-19 wt %, preferably about 12-18 wt %, and more preferably about 14-17 wt %. In any embodiment of the present invention, the nutritional composition contains protein, fat and carbohydates in amounts of:
protein: to provide 20-53% of total energy of the composition,
fat: to provide 18-36% of the total energy of the composition, and
carbohydrates (excluding fibre): to provide 4-61% of the total energy of the composiiton

The nutritional composition preferably contains fibre (i.e. at least PHGG and AG) to provide 1-7% of the total energy of the composition.

The combinations of the present invention are particularly suitable for preparing nutritional composition suitable for a low FODMAP diet. Preferably, the nutritional composition for low FODMAP diet provides 0.5 g or less, preferably 0.4 g or less, and more preferably 0.3 g or less of fermentable oligo-, di- and mono-saccharides and polyols (FODMAPS) per serving. Advantageously, the combination of AG and PHGG of the present invention provides a source of soluble fibres. Soluble fibres are particularly useful for alleviating symptoms of IBS. In particular, soluble fibres have the effect of increasing the water content and bulk of alimentary contents, and hence can normalize the progression of stool through the intestines. Thus, soluble dietary fibre improves the regularity of bowel movements, and contributes to the generation of soft stools, and hence improves symptoms of constipation and bowel movement pain. Further such fibres can delay gastric emptying, and hence reduce the symptoms of diarrhoea.

Nutritional composition according to any embodiment or aspect of the present invention may further comprise one or more micronutrients. Such micronutrients include those selected from the group consisting of vitamins, minerals and trace elements.

A nutritional composition according to any aspect or embodiment of the present invention may further comprise one or more probiotics as described above. When a probiotic is included in the nutritional composition, the composition may be in the form of a liquid, or preferably, a powder.

Nutritional compositions of the present invention may also include one or more dietary or pharmaceutically acceptable excipients. The compositions may preferably be form of a liquid, such as a ready-to-drink liquid. The liquid is preferably aqueous-based, i.e. the solid components in the composition, i.e. AG, PHGG and optionally maltodextrin, along with the remaining solid components when present, including fat, protein, carbohydrate, micronutrients, and excipients, are present in an aqueous solution or suspension.

The nutritional composition is preferably in the form of a liquid. The nutritional composition may further comprise at least one pharmaceutically or dietary acceptable additive or excipient, for example selected from: stabilizers, emulsifiers, surfactants, solubilising agents, buffers, wetting agents, carriers, antioxidants, preservatives, flavouring agents, sweeteners and dyes.

Alternatively, the nutritional composition may be in the form of a solid, preferably in the form of a powder. The powder may be in a form suitable for aqueous reconstitution.

In the solid or powder formulations containing the combination of AG and PHGG, such as a powder for aqueous reconstitution, the amount of AG and PHGG is preferably about 2 to about 18 wt %, about 2 to about 15 wt %, about 3 to about 15 wt %, about 4 to about 10 wt % or about 5 to about 9 wt %. The solid or powder composition may further comprising a starch hydrolysate, preferably wherein the starch hydrolysate is maltodextrin. The amount of starch hydrolysate, preferably maltodextrin, is preferably: about 5 to about 45 wt %, about 5 to about 45 wt %, about 10 to about 35 wt %, about 10 to about 30 wt % or about 10 to about 28 wt %. The solid/powder nutritional composition preferably includes macronutrients, preferably protein, fat and carbohydrate. The amount of carbohydrates in the solid nutritional composition is: preferably about 5 to about 90 wt %, about 10 to about 80 wt %, about 15 to about 75 wt %, about 15 to about 70 wt % or about 20 to about 65 wt %. Preferably, the amount of carbohydrates in the form of sugars in the solid nutritional composition is: about 2 to about 55 wt %, about 3 to about 50 wt %, about 3 to about 45 wt %, about 3.5 to about 35 wt % or about 3 to about 30 wt %. Preferably, the amount of protein in the solid nutritional composition is: about 18 to about 75 wt %, about 20 to about 70 wt %, about 22 to about 65 wt % or about 22 to about 55 wt %. Preferably, the amount of fat in the solid nutritional composition is: about 4 to about 25 wt %, about 4 to about 23 wt %, about 4 to about 16 wt %, about 4 to about 14 wt % or about 5 to about 10.5 wt %. The solid/powder nutritional composition is preferably suitable for a low FODMAP diet, In particular, the solid nutritional composition preferably provides less than 0.5 g or less, preferably 0.4 g or less, and more preferably 0.3 g or less of fermentable oligo-, di- and mono-saccharides and polyols (FODMAPS) per serving). The solid nutritional composition may include one or more micronutrients selected from the group consisting of vitamins, minerals and trace elements, and/or one or more probiotics as described above. The solid nutritional compositions of the present invention may further comprising one or more dietary or pharmaceutically acceptable excipients. For example the solid nutritional composition may further comprise at least one pharmaceutically or dietary acceptable additive or excipient, for example selected from: stabilizers, emulsifiers, surfactants, solubilising agents, buffers, wetting agents, carriers, antioxidants, preservatives, flavouring agents, sweeteners and dyes.

Solid nutritional compositions of the present invention as described above are preferably in the form of powder for reconstitution (preferably in water) into a liquid nutritional composition. The liquid composition including the combination of AG and PHGG has an enhanced or improved mouthfeel.

The present invention further provides method of promoting gastrointestinal microbiota balance and health comprising administering an effective amount of a nutritional composition according to any aspect or embodiment of the present invention as described herein to an individual in need of such treatment.

The present invention also encompasses a nutritional composition according to any aspect or embodiment of the present invention as described herein for the treatment or prophylaxis of symptoms of a GI tract or a digestive disorder, preferably selected from: impaired GI motility, dyspepsia, abdominal pain, abdominal cramp, diarrhoea, constipation, disturbed bowel habits, bloating, excess gas, abdominal discomfort and abdominal distension.

The present invention also provides a nutritional composition according to any aspect or embodiment of the present invention as described herein for the treatment or prophylaxis of symptoms of a GI tract or a digestive disorder, preferably selected from: impaired GI motility, dyspepsia, abdominal pain, abdominal cramp, diarrhoea, constipation, disturbed bowel habits, bloating, excess gas, abdominal discomfort and abdominal distension.

A nutritional composition according to the present invention may be prepared in any suitable manner. For example, it may be prepared by blending together the protein, the carbohydrate source, and the fat source (if these are to be included) in appropriate proportions. AG and PHGG and optionally maltodextrin, may be added at this point. The vitamins and minerals may be added at this point but are usually added later to avoid thermal degradation. Any lipophilic vitamins, emulsifiers and the like may be dissolved into the fat source prior to blending. Water, preferably water which has been subjected to reverse osmosis, may then be mixed in to form a liquid mixture. The temperature of the water is conveniently about 50° C. to about 80° C. to aid dispersal of the ingredients. Commercially available liquefiers may be used to form the liquid mixture. The liquid mixture is then homogenised; for example in two stages. The liquid mixture can be heat treated for sterilization or pasteurization to reduce bacterial loads. For example, the mixture can be UHT treated, e.g. by rapidly heating the liquid mixture to a temperature in the range of about 80° C. to about 150° C. for about 5 seconds to about 5 minutes (e.g. by steam injection, or by heat exchanger; for example a plate heat exchanger.

Then, the liquid mixture may be cooled to about 60° C. to about 85° C.; for example by flash cooling. The liquid mixture may then be again homogenised; for example in two stages at about 2000 psi to about 5000 psi (preferably about 3000 psi to about 4000 psi, more preferably about 3500 to about 4000 psi) in the first stage and about 400 to about 1000 psi (preferably about 500 to about 900 psi, more preferably about 600 to about 800 psi) in the second stage.

The liquid nutritional composition may then be packaged into suitable containers, for example to provide a single serving container. Alternatively, if the mixture is sterilized by autoclave treatment, the liquid mixture (e.g. after homogenization) can be packaged and subjected to autoclave treatment in the package.

Alternatively, a powder for reconstitution into a liquid nutritional composition may be prepared by combining a mixture comprising PHGG and GA, and nutritional components including carbohydrate, fat and protein), and optionally vitamins, minerals and trace elements, and pharmaceutically acceptable excipients. The powder may be packaged into single serving sachets, or into a carton for dispensing multiple servings. A nutritional composition in the form of a beverage, may be prepared by reconstituting the powder with water.

The invention will now be further illustrated by reference to the following non-limiting examples.

### EXAMPLES

### Example 1—Mouthfeel Enhancement Using AG and PHGG

Nine prototype formulations were prepared. Of these, 6 contained maltodextrin in combination with varying gums, 2 contained corn syrup and varying gums, and one formulation was a high FODMAP variant.

The variants were compared to two references for “thickness” and “body”. The low solids reference was composed of: corn syrup (no added fiber or maltodextrin) it also had milk protein concentrate, caseinates, blend of corn, canola and sunflower oils.

The high solids reference is a commercially available high protein formulation, BOOST® High Protein Drink [containing in a 237 ml serving: 15 g of protein (milk protein concentrate, sodium caseinate, calcium caseinate and soy protein isolate), total carbohydrates: 33 g (27 g of which are sugars), 6 g of total fat (blend of corn, canola and high oleic sunflower oils), and vitamins and minerals (<0.5%)—see https://www.boost.com/products/high-protein]. The high solids reference contains no added fibre or maltodextrin).

The high FODMAP variant contains hydrolysed inulin (a FructoOligoSaccharide (FOS) Inulin), which is commercially available, and which is a powder prepared by partial enzymatic hydrolysis of chicory inulin and subsequent concentration. The product contains mainly oligofructose with small quantities of fructose, glucose and sucrose. The oligofructose component consists of a mixture of oligosaccharides, which are polymers of D-fructose units linked via β(2-1) linkages. Some of these polymers are terminated by a D-glucose unit linked by α(1-2) linkage. The total number of fructose or glucose units of oligofructose (Degree of Polymerization=DP) ranges mainly between 2 and 60, with an average DP of >10.

To prepare each prototype formulation, the fibres (AG and/or PHGG) are added to the low solids reference. The resulting formulations of variants 1-9 are as follows:

| | | | | | | | | | | High solids |
|---|---|---|---|---|---|---|---|---|---|---|
| | | | | | | | | | | ref. (BOOST ® |
| VARIANT | 1 | 2 | 3 | 4 | 5 | 6 | 7 | 8 | 9 | high protein) |
| Gum Arabic (gm/serving) | | 1.5 | 3 | 3 | 1.5 | 1.5 | 3 | 6 | 6 g | — |
| | | | | | | | | | FOS | |
| PHGG (gm/serving) | 6 | 3 | 3 | 3 | 1.5 | 1.5 | 1.5 | — | — | — |
| Corn syrup | — | — | — | yes | — | yes | | — | — | — |
| Milk protein | 4.14 | 4.14 | 4.14 | 4.14 | 4.14 | 4.14 | 4.14 | 4.14 | 4.14 | 4.14 |
| concentrate | | | | | | | | | | |
| 85% Low Lactose | | | | | | | | | | |
| PHGG | 2.84 | 1.42 | 1.42 | 1.42 | 0.68 | 0.68 | 0.71 | — | — | — |
| Gum Acacia | | 0.72 | 1.43 | 1.43 | 0.69 | 0.69 | 1.43 | 2.86 | — | — |
| FOS | — | — | — | — | — | — | — | — | 2.6 | — |
| (fructooligosaccharides) | | | | | | | | | | |
| Maltodextrin | 2.71 | 3.47 | 2.85 | | 4.16 | | 3.53 | 2.96 | 2.87 | |
| Glucose Syrup | — | — | — | 4.15 | — | 6.2 | — | — | — | 5.881 |
| Sugar | 2.15 | 2.15 | 2.15 | 2.15 | 2.15 | 2.15 | 2.15 | 2.15 | 2.15 | 2.15 |
| Oil Mix (Corn, canola | 1.51 | 1.51 | 1.51 | 1.51 | 1.51 | 1.51 | 1.51 | 1.51 | 1.51 | 2.21 |
| and sunflower oils) | | | | | | | | | | |
| Soy Protein Powder | 1.00 | 1.00 | 1.00 | 1.00 | 1.00 | 1.00 | 1.00 | 1.00 | 1.00 | 1.00 |
| Calcium caseinate | 1.90 | 1.90 | 1.90 | 1.90 | 1.90 | 1.90 | 1.90 | 1.90 | 1.90 | 1.90 |
| Soy lecithin liquid | 0.056 | 0.056 | 0.056 | 0.056 | 0.056 | 0.056 | 0.056 | 0.056 | 0.056 | 0.082 |

Water to 100% w/w

Each of the nine prototype formulations were compared to the low solid reference and to the high solid reference.

Sensory evaluation was determined by panel (n=30), where the panellists were trained on sensory attributes.

N=30 for each panel, panelists trained on sensory attributes

Panelists asked to ID which sample within the pair had more thickness/body

Thickness and body evaluated separately for each comparison

For the nine compositions in each of the “low” and “high” solids references, the panellists were asked to identify which samples (i.e. “low” or “high” solids) had more “thickness” and “body”.

The results are presented in the table below:

| | | Thickness | | | | Body | | | | Analytical Data | |
|---|---|---|---|---|---|---|---|---|---|---|---|
| | | Low | | High | | Low | | High | | | Total |
| | | Solids | | Solids | | Solids | | Solids | | Viscosity | Solids |
| Variant | Description | Reference¹ | | Reference¹ | | Reference¹ | | Reference¹ | | Centipoise³ | wt %? |
| 1 | 6 g PHGG | 28 | (2) | 25 | (5) | 29 | (1) | 20 | (10) | 37.2 | 16.63 |
| | with | | | | | | | | | | |
| | maltodextrin | | | | | | | | | | |
| 2 | 1.5 g AG | 25 | (5) | 19 | (11) | 30 | (0) | 19 | (11) | 30.5 | 16.71 |
| | 3 g PHGG | | | | | | | | | | |
| | with | | | | | | | | | | |
| | maltodextrin | | | | | | | | | | |
| 3 | 3 g AG | 27 | (3) | 24 | (6) | 28 | (2) | 25 | (5) | 39.8 | 16.67 |
| | 3 g PHGG | | | | | | | | | | |
| | with | | | | | | | | | | |
| | maltodextrin | | | | | | | | | | |
| 4 | 3 g AG | 25 | (4)² | 23 | (6)² | 29 | (1) | 23 | (7) | 40.1 | 16.79 |
| | 3 g PHGG- | | | | | | | | | | |
| | with | | | | | | | | | | |
| | corn syrup | | | | | | | | | | |
| 5 | 1.5 g AG | 27 | (3) | 21 | (8)² | 22 | (8) | 18 | (12) | 28.5 | 16.75 |
| | 1.5 g PHGG | | | | | | | | | | |
| | with | | | | | | | | | | |
| | maltodextrin | | | | | | | | | | |
| 6 | 1.5 g AG | 22 | (8) | 8 | (22) | 20 | (10) | 12 | (18) | 26.9 | 16.68 |
| | 1.5 g PHGG- | | | | | | | | | | |
| | with corn | | | | | | | | | | |
| | syrup | | | | | | | | | | |
| 7 | 3 g AG | 28 | (2) | 23 | (7) | 29 | (1) | 21 | (9) | 40.9 | 16.74 |
| | 1.5 g PHGG | | | | | | | | | | |
| | with | | | | | | | | | | |
| | maltodextrin | | | | | | | | | | |
| 8 | 6 g AG | 29 | (1) | 24 | (6) | 28 | (2) | 21 | (9) | 55 | 16.69 |
| | with | | | | | | | | | | |
| | maltodextrin | | | | | | | | | | |
| 9 | 6 g FOS | 19 | (11) | 5 | (25) | 19 | (11) | 5 | (25) | 17.8 | 16.21 |
| | with | | | | | | | | | | |
| | maltodextrin | | | | | | | | | | |

| | | | | | | | |
|---|---|---|---|---|---|---|---|
| REF | Low solids | — | — | — | — | 16.2 | 16.53 |
| | reference | | | | | | |
| REF | High solids | — | — | — | — | 20 | 22.92 |
| | reference | | | | | | |

¹The first number represents the number of participants who selected the variant. The numbers in brackets represent the number of participants who selected the reference. Figures in bold represent statistically different results.
²N = 29 participants.
³Measured using a Brookfield viscometer with spindle 1 at 60 rpm, at a product temperature of 25° C.

The results demonstrate that, as predicted, a reduction of solids (cf. Low solids reference vs High solids reference) results in a significant loss of thickness and a detrimental impact on mouthfeel. The addition of a combination of PHGG and AG to the low solids reference enhances or provides a comparable mouthfeel to the high solids reference.

Moreover, this effect was surprisingly seen even when using a lower total amount of the combination of PHGG and AG, compared with the same amount of a single fibre (i.e. PHGG or AG alone). Advantageously, this allows a reduction of fibre additives required to achieve an acceptable mouthfeel, and may be important if dietary guidelines require an upper limit for a particular fibre. Since the combination of PHGG with AG in the compositions of the present invention enable the same or better mouthfeel enhancement at a lower amount compared to the individual components (cf variant 2 vs variant 5), it is possible to control the composition in order to meet with upper limits stipulated by regulation guidelines. Moreover, from the point of view of PHGG, which is particularly expensive, by adding AG to the PHGG, the combination of the present invention enables a reduction in the amount of PHGG to achieve the same, or better mouthfeel enhancement, in a more cost-effective manner.

These results are particularly surprising in view of literature reports that PHGG as a food additive does not alter the rheology and texture of final products (Yoon, S.-J. et al.: Journal of Clinical Biochemistry and Nutrition 2008, 42, 1-7). Moreover, Heini, A. F. et al.: (International Journal of Obesity 1998, 22, 906-909), reported that PHGG can be added as a soluble fibre to a nutritional drink without the subject's conscious awareness—hence it had no effect on mouthfeel or texture—such that double blind trials could be conducted.

As shown by variants 5 and 6, the addition of maltodextrin (variant 5) enhanced mouthfeel (thickness and body) compared to corn syrup. In particular, the maltodextrin variant 5 was perceived to be thicker than the high solids variant, whereas the corn syrup variant 6 wans significantly perceived to be less thick than the high solids reference. These results further indicate that these results relate to a true enhancement of mouthfeel, rather than a mere perception of increased viscosity, because the viscosities of variants 5 and 6 are very close (28.5 vs 26.9 centipoise).

The low fibre (i.e. low AG/PHGG) variants show better results compared to performance of FOS which was used at a higher level. Thus, these low fibre blends provide a method by which fibre (in the form of soluble fibres, AG and PHGG) can be added particularly to low FODMAP supplements, whilst advantageously enhancing the mouthfeel.

The addition of fructooligosaccharides alone, although enables fortification with fibre, without increasing viscosity, does not result in a mouthfeel enhancement. Moreover, FOS are unsuitable as fibre sources for a low FODMAP diet.

### Example 2

A nutritional supplement beverage containing the following ingredients can be prepared by conventional processes:

| Ingredient | wt % |
|---|---|
| Partially hydrolysed guar gum | 0.5 |
| powder | |
| Gum acacia | 1.0 |
| Maltodextrin DE10-20 | 3.7 |
| Milk protein concentrate, | 7 |
| sodium or potassium | |
| caseinate | |
| Liquid sugar | 2.8 |
| Vegetable oil (canola oil) | 1.2 |
| Vitamins | 0.3 |
| Minerals and trace elements | 1.1 |
| Emulsifiers | 0.1 |
| Flavourings | 0.3 |
| Water | 82 |

### Example 3

A nutritional supplement beverage containing the following ingredients can be prepared by conventional processes:

| Ingredient | wt % |
|---|---|
| Partially hydrolysed guar gum | 0.35 |
| powder | |
| Gum acacia | 1 |
| Maltodextrin demin powder | 3.8 |
| DE 10-20 | |
| Sugar | 2 |
| Milk Protein Concentrate and | 7.4 |
| Soy Protein | |
| Vegetable oil (corn oil) | 1.2 |
| Vitamins | 0.3 |
| Minerals and trace elements | 1.3 |
| Emulsifiers | 0.25 |
| Flavourings | 0.3 |
| Water | 82.1 |

### Example 4

A powder for reconstitution into a nutritional composition containing the following can be prepared by conventional processes:

| Ingredient | wt % |
|---|---|
| Partially hydrolysed guar gum | 2.7 |
| powder | |
| Gum acacia | 5.2 |
| Maltodextrin | 27 |
| Carbohydrate (sugars) | 12 |
| Soy powder, 90% | 5.7 |
| Milk protein concentrate | 24 |
| Sodium caseinate | 5.5 |
| Calcium caseinate | 5.6 |
| Canola and sunflower oils | 8.6 |
| Silicone emulsion, liquid 20% | 0.03 |
| Vitamins | 0.22 |
| Minerals | 0.8 |
| Sweetener (sucralose) | 0.35 |
| Emulsifiers | 0.6 |
| (carrageenan, and gum blend) | |
| Flavourings | 1.7 |

The powder can packaged into single serving sachets (e.g. containing 25 g of powder), or into multi-serving cartons. A nutritional supplement drink can be prepared by reconstituting a single serving of powder using water (e.g. 25 g of powder made up to a volume of 180 ml).

1. A combination of acacia gum (AG) and partially hydrolysed guar gum (PHGG) for improving mouthfeel of a nutritional composition, wherein AG and PHGG are present in a weight ratio of AG:PHGG of about 5:1 to about 1:5.

2. A combination according to claim 1 wherein AG and PHGG are present in a weight ratio of AG:PHGG of about 5:1 to about 1:3.

3. A combination according to claim 1, wherein the amount of AG and PHGG is: about 0.6 to about 2.5 wt % of the nutritional composition.

4. A combination according to claim 1, further comprising a starch hydrolysate.

5. A combination according to claim 4, wherein the ratio of starch hydrolysate, preferably maltodextrin, to total AG and PHGG is: about 5:1 to about 1:5.

6. A combination according to claim 1, wherein the nutritional composition has an energy density of less than about 1 kcal/g.

7. A combination according to claim 1, wherein the nutritional composition has a total solids content of: about 12 to about 20 wt %.

8. A combination according to claim 1, wherein the nutritional composition comprises protein, fat and carbohydrate.

9. A combination according to claim 8, wherein the amount of solids from the protein, fat and carbohydrate in the nutritional composition is about 12 to about 20 wt %.

10. A combination according to claim 8, wherein the amount of carbohydrates in the nutritional composition is: about 10-17 wt %.

11. A combination according to claim 8, wherein the amount of carbohydrates in the form of sugars in the nutritional composition is: about 7 wt % or less.

12. A combination according to claim 8, wherein the amount of protein in the nutritional composition is about 4 to about 24 wt %.

13. A combination according to claim 8, wherein the amount of fat in the nutritional composition is less than about 4 wt %.

14. A combination according to claim 1, wherein the nutritional composition provides 0.5 g or less of fermentable oligo-, di- and mono-saccharides and polyols (FODMAPS) per serving.

15. A combination according to claim 1, wherein the nutritional composition is a liquid.

16-17. (canceled)

18. A nutritional composition comprising fat, protein carbohydrate, and a combination of acacia gum (AG) and partially hydrolysed guar gum (PHGG) wherein AG and PHGG are present in a weight ratio of AG:PHGG of about 5:1 to about 1:5, and wherein the nutritional composition has a total solids content of: about 12 to about 20 wt %.

19-53. (canceled)

54. A method for achieving a goal selected from the group consisting of promoting GI microbiota balance and health treatment or prophylaxis of a functional GI disorder or a digestive disorder comprising administering an effective amount of a nutritional composition comprising fat, protein carbohydrate, and a combination of acacia gum (AG) and partially hydrolysed guar gum (PHGG) wherein AG and PHGG are present in a weight ratio of AG:PHGG of about 5:1 to about 1:5, and wherein the nutritional composition has a total solids content of: about 12 to about 20 wt % to an individual in need of such treatment.

55-58. (canceled)
